(19) United States
(12) Patent Application Publication
(10) Pub. No.: US 2025/0205291 A1
(43) Pub. Date: Jun. 26, 2025

(54) METHODS AND COMPOSITIONS FOR SLOWING DIABETES DEVELOPMENT OF REDUCING A RISK OF DIABETES

(71) Applicant: Abbott Laboratories, Abbott Park, IL (US)

(72) Inventors: José María López Pedrosa, Cenes de la Vega, Granada (ES); Manuel Manzano, Dilar Granada (ES); Ricardo Rueda Cabrera, Granada, Granada (ES)

(21) Appl. No.: 18/851,570

(22) PCT Filed: Apr. 21, 2023
(86) PCT No.: PCT/US2023/066047

§ 371 Date: Sep. 26, 2024

Related U.S. Application Data
(60) Provisional application No. 63/333,170, filed on Apr. 21, 2022.

(51) Int. Cl. A61K 35/745; A23L 33/125; A23L 33/135; A23L 33/21; A61K 31/7004; A61K 31/7016; A61K 31/718; A61P 3/10

(52) U.S. Cl. CPC A61K 35/745; A23L 33/125; A23L 33/135; A23L 33/21; A61K 31/7004; A61K 31/7016; A61K 31/718; A61P 3/10

(57) ABSTRACT

A method for slowing diabetes development in a diabetic or prediabetic individual or reducing a risk of diabetes development in an individual at risk of developing diabetes comprises administering Bifidobacterium animalis subsp. lactis CECT 8145 (BPL1) and a carbohydrate blend to the individual. The carbohydrate blend comprises a source of at least one carbohydrate that provides rapidly available glucose, a source of at least one carbohydrate that provides slowly available glucose, and a source of at least one non-digestible carbohydrate or resistant starch. A nutritional composition comprises protein, fat, the carbohydrate blend, and BPL1.

### FIELD OF THE INVENTION

The present invention is directed to methods and compositions suitable for slowing diabetes or prediabetes development or reducing a risk of diabetes development, for example in an individual at risk of developing diabetes.

### BACKGROUND OF THE INVENTION

The intestinal microbiome (the microorganisms of the intestine) play an important role in host metabolism, and the importance of the intestinal microbiome has gained wide scientific interest in health and disease. Emerging evidence suggests a causal link between microbial dysbiosis and human diseases such as obesity and diabetes. Several studies have confirmed reduced gut microbial diversity and altered microbiota composition in adults with obesity and diabetes compared with healthy individuals. The intestinal microbiome composition is also susceptible to nutritional changes or medication. Therefore, modification of the intestinal bacterial composition towards a “healthier” microbiome has become attractive as a possible therapeutic approach.

Probiotics are defined as live microorganisms that, when administered in adequate amounts, confer a health benefit on the host. Postbiotics are defined as preparations of inanimate microorganisms and/or their components that confer a health benefit on the host. They include any substance released or produced through the metabolic activity of microorganisms, which has a direct or indirect beneficial effect for the host. One known probiotic is Bifidobacterium animalis subsp. lactis CECT 8145 (BPL1), supplied by Archer Daniels Midland Company. BPL1, both live (probiotic) and inanimate (postbiotic), has been disclosed as providing a beneficial effect on the management of metabolic alterations associated with obesity and related diseases including metabolic syndrome, hypertension, glycemia, and type 2 diabetes. See WO 2015/007941. Different works have described the beneficial effects of the postbiotic heat killed or heat treated BPL1 (hkBPL1 or BPL1 HT) on obesity by different mechanisms of actions that involve: (i) reduction in body weight and mesenteric adiposity with a parallel increase in lean body mass, (ii) stimulation of energy expenditure, and (iii) improvements in insulin sensitivity as well as dyslipidemia.

However, further improvements in the use of probiotics and/or postbiotics for obtaining and/or maintaining health benefits are needed.

### SUMMARY OF THE INVENTION

Accordingly, it is an object of the present invention to provide additional improvement in providing health benefits from probiotic or postbiotic BPL1.

In one embodiment, the invention is directed to a method for slowing diabetes development in a diabetic or prediabetic individual or reducing a risk of diabetes development in an individual at risk of developing diabetes. The method comprises administering Bifidobacterium animalis subsp. lactis CECT 8145 (BPL1) and a carbohydrate blend to the individual. The carbohydrate blend comprises a source of at least one carbohydrate that provides rapidly available glucose, a source of at least one carbohydrate that provides slowly available glucose, and a source of at least one non-digestible carbohydrate or resistant starch.

In another embodiment, the invention is directed to a nutritional composition comprising protein, fat, a carbohydrate blend, and BPL1, wherein the carbohydrate blend comprises a source of at least one carbohydrate that provides rapidly available glucose, a source of at least one carbohydrate that provides slowly available glucose, and a source of at least one non-digestible carbohydrate or resistant starch.

The methods and compositions of the invention are advantageous in managing metabolic alterations associated with diabetes development and progression in a manner that slows diabetes or prediabetes development or reduces a risk of diabetes development in an individual, for example in an individual at risk of developing diabetes. These and additional advantages will be more fully apparent in view of the following detailed description.

### BRIEF DESCRIPTION OF THE DRAWINGS

The detailed description of the invention is more fully understood in view of the drawings, in which:

FIG. 1 shows total fat mass in groups of insulin-resistant obese rats resulting from 4 weeks of feeding with different diets, including an OB group, which was fed a high fat diet, an OB+BPL1 HT group, which was fed the high fat diet supplemented with post-biotic (heat-killed) Bifidobacterium animalis subsp. lactis CECT 8145 (BPL1 HT), an OB+CBLEND group which was fed the high fat diet supplemented with a carbohydrate blend according to the invention (CBLEND), and an OB+BPL1 HT+CBLEND group, which was fed the high fat diet supplemented with both BPL1 HT and CBLEND, and as compared with a control group fed a lean diet (Lean), as described in Example 1;

FIG. 2 shows glucagon-like peptide 1 (GLP-1) secretion in the groups of insulin-resistant obese rats in the OB group, the OB+BPL1 HT group, the OB+CBLEND group, and the OB+BPL1 HT+CBLEND group, after 4 weeks of feeding with the different diets, and as compared with the Lean group, as described in Example 1;

FIG. 3 shows short-chain fatty acids (SCFAs) detected in cecum of the OB group, the OB+BPL1 HT group, the OB+CBLEND group, and the OB+BPL1 HT+CBLEND group, after 4 weeks of feeding with the different diets, and as compared with the Lean group, as described in Example 1;

FIG. 4A shows glucose levels detected in blood after an oral glucose challenge in groups of diabetic rats, i.e., insulin-resistant obese rats treated with streptozotocin (STZ), which causes initial beta cell dysfunction and subsequent hyperglycemia and therefore simulates type 2 diabetes evolution, after 4 weeks of feeding with different diets, including a DM group, which was fed a high fat diet, a DM+BPL1 HT group, which was fed the high fat diet supplemented with BPL1 HT, a DM+CBLEND group which was fed the high fat diet supplemented with CBLEND, and a DM+BPL1 HT+CBLEND group, which was fed the high fat diet supplemented with both BPL1 HT and CBLEND, and as compared with a control group fed a lean diet (Lean), as described in Example 2;

FIG. 4B shows AUC of blood glucose in the DM group, the DM+BPL1 HT group, the DM+CBLEND group, and the DM+BPL1 HT+CBLEND group of diabetic rats, and as compared with the Lean group, as described in Example 2; and

FIG. 5 shows HbA1c levels detected in blood in the DM group, the DM+BPL1 HT group, the DM+CBLEND group, and the DM+BPL1 HT+CBLEND group of diabetic rats, and as compared with the Lean group, as described in Example 2.

The drawings are provided to illustrate certain features of the invention and are not to construed as limiting the embodiments or the scope of the invention.

### DETAILED DESCRIPTION

While the general inventive concepts are susceptible of embodiment in many different forms, described herein in detail are specific embodiments of the invention, with the understanding that the present disclosure is to be considered as an exemplification of the principles of the general inventive concepts. Accordingly, the general inventive concepts are not intended to be limited to the specific embodiments illustrated and described herein.

All percentages, parts and ratios as used herein, are by weight of the total composition, unless otherwise specified. All such weights as they pertain to listed ingredients are based on the active level and, therefore, do not include solvents or byproducts that may be included in commercially available materials, unless otherwise specified.

The terminology as set forth herein is for description of the embodiments only and should not be construed as limiting the disclosure as a whole. Unless otherwise specified, “a,” “an,” “the,” and “at least one” are used interchangeably. Furthermore, as used in the description and the appended claims, the singular forms “a,” “an,” and “the” are inclusive of their plural forms, unless the context clearly indicates otherwise.

Throughout this specification, when a range of values is defined with respect to a particular characteristic of the present invention, the present invention relates to and explicitly incorporates every specific subrange therein. Additionally, throughout this specification, when a group of substances is defined with respect to a particular characteristic of the present invention, the present invention relates to and explicitly incorporates every specific subgroup therein. Any specified range or group is to be understood as a shorthand way of referring to every member of a range or group individually as well as every possible subrange or subgroup encompassed therein.

The methods and nutritional compositions described herein may comprise, consist of, or consist essentially of the essential steps and elements, respectively, as described herein, as well as any additional or optional steps and elements, respectively, described herein. Any combination of method or process steps as used herein may be performed in any order, unless otherwise specified or clearly implied to the contrary by the context in which the referenced combination is made.

The various embodiments of the nutritional compositions of the invention may also be substantially free of any optional or selected ingredient or feature described herein, provided that the remaining nutritional composition still contains all of the required ingredients or features as described herein. In this context, and unless otherwise specified, the term “substantially free” means that the selected nutritional composition contains less than a functional amount of the optional ingredient, typically less than 1%, including less than 0.5%, including less than 0.1%, and also including zero percent, by weight, of such optional or selected essential ingredient.

Unless otherwise indicated herein, all exemplary embodiments, sub-embodiments, specific embodiments and optional embodiments are respective exemplary embodiments, sub-embodiments, specific embodiments and optional embodiments to all embodiments described herein.

In a first embodiment, the method is directed to a method for slowing diabetes development in a diabetic or prediabetic individual or reducing a risk of diabetes development in an individual at risk of developing diabetes. Diabetes is a chronic disease that occurs either when the pancreas does not produce enough insulin or when the body cannot effectively use the insulin it produces. Insulin is a hormone that regulates blood glucose. Hyperglycemia, also referred to as raised blood glucose or raised blood sugar, is a common effect of uncontrolled diabetes and over time leads to serious damage to many of the body's systems. Type 2 diabetes (also referred to as non-insulin-dependent, or adult-onset diabetes, although it can also occur in children and juveniles) results from the body's ineffective use of insulin. A majority of people with diabetes have type 2 diabetes, which is often the result of excess body weight and physical inactivity. Prediabetes is a serious health condition where blood glucose (sugar) levels are higher than normal, but not high enough to be diagnosed as type 2 diabetes. In some specific cases, a prediabetic individual is insulin resistant, i.e., the individual's cells do not respond normally to insulin and as a result, glucose cannot enter the cells as readily. Consequently, glucose levels in the blood increase. This can eventually lead to type 2 diabetes. Insulin resistance typically has no symptoms. In fact, many people with prediabetes have no symptoms, and without intervention, are likely to develop type 2 diabetes.

Therefore, in specific embodiments, the individual exhibits prediabetes or diabetes and the method of the invention slows diabetes development. In other specific embodiments, the individual is at risk of developing diabetes, and, more specifically, is obese and/or exhibits insulin resistance, and the method of the invention reduces a risk of diabetes development in the individual.

The methods of the invention comprise administering both BPL1 and a defined carbohydrate blend. The inventors have discovered that a diet with both BPL1 and the carbohydrate blend surprisingly and unexpectedly reduces the occurrence of metabolic activity leading to diabetes and prediabetes development and to an extent greater than that obtained with administration of either BPL1 or the carbohydrate blend individually. The methods therefore effectively manage metabolic alterations associated with diabetes development and progression in a manner that slows diabetes or prediabetes development or reduces a risk of diabetes development in an individual at risk of developing diabetes.

The BPL1 employed in the invention may be probiotic, i.e., in a live form, or postbiotic, i.e., in inanimate form. Typically, postbiotic BPL1 has been heat treated to eliminate live cells. Surprisingly, as shown in the examples, heat-treating BPL1 to render it postbiotic does not prevent the advantageous benefits of administration together with the carbohydrate blend. BPL1 is commercially available and may be obtained, for example, from Archer Daniels Midland Company, Chicago, IL, USA, or BPL1 may be prepared as described by Caimari et al., Journal Functional Foods (2017), 38: 251-63, incorporated herein by reference.

Within the context of the present invention, the carbohydrate blend comprises a source of at least one carbohydrate that provides rapidly available glucose, a source of at least one carbohydrate that provides slowly available glucose, and a source of at least one non-digestible carbohydrate or resistant starch. Carbohydrates that provide rapidly available glucose are rapidly absorbed in the duodenum and proximal regions of the small intestine, leading to a rapid elevation of blood glucose and usually a subsequent episode of hypoglycemia. Carbohydrates that provide slowly available glucose are steadily but completely digested, resulting in prolonged glucose release from the lumen of the small intestine into the blood stream. Non-digestible carbohydrates or resistant starches are carbohydrates or factions thereof that are not digested in the upper gastrointestinal tract, but are fermented in the large intestine by the gut microbiota, producing short chain fatty acids that advantageously provide additional energy to the body.

The terms “rapidly available glucose” and “slowly available glucose” as used herein reflect the rate at which glucose becomes available for absorption in the human small intestine according to the in vitro method developed by Englyst et al. Am J Clin Nutr (1999), Vol. 69, pp 448-454, which is known in the art and incorporated by reference herein. This in vitro method characterizes dietary carbohydrates with regard to their chemical composition and likely gastrointestinal fate. The glycemic carbohydrate fraction that is available for absorption in the small intestine is measured as the sum of sugars and starch (including maltodextrins) and excludes digestion-resistant starch. The Englyst method determines rapidly available glucose, slowly available glucose, and starch fractions by measuring the amount of glucose released from a carbohydrate or carbohydrate source during timed incubations (20 minutes and 120 minutes) with digestive enzymes under standardized conditions. In accordance with the Englyst method, for a carbohydrate or carbohydrate source, the amount of glucose measured at 20 minutes (G20) represents “rapidly available glucose,” whereas the difference between the amount of glucose measured at 120 minutes (G120) and the G20 value, i.e., G120-G20, represents “slowly available glucose.” The Englyst method also enables the calculation of: (i) rapidly digestible starch, which contributes to the amount of rapidly available glucose; (ii) slowly digestible starch, which contributes to the amount of slowly available glucose; (iii) total starch; and (iv) resistant starch.

In specific embodiments, the source of at least one carbohydrate that provides rapidly available glucose comprises (i) a monosaccharide; (ii) a glucose unit and a fructose unit joined by an α-1, β-2 glycosidic linkage; (iii) a glucose unit and a galactose unit joined by a β (1,4) glycosidic linkage; (iv) glucose units joined by α (1,4) glycosidic linkages; (v) glucose units joined by α (1,6) glycosidic linkages; or (vi) an oligosaccharide having a random mixture of α (1,2), α (1,3), α (1,4), and α (1,6) glycosidic linkages, or a combination of two or more thereof.

In more specific embodiments, the source of at least one carbohydrate that provides rapidly available glucose in the carbohydrate blend comprises a monosaccharide. Exemplary monosaccharides suitable for use in the carbohydrate blend to provide rapidly available glucose include, but are not limited to, glucose, fructose, tagatose, galactose, mannose, and ribose.

In further specific embodiments, the source of at least one carbohydrate that provides rapidly available glucose in the carbohydrate blend comprises a glucose unit and a fructose unit joined by an α-1, β-2 glycosidic linkage. One example of a carbohydrate that includes a glucose unit and a fructose unit joined by an α-1, β-2 glycosidic linkage is sucrose.

In additional specific embodiments, the source of at least one carbohydrate that provides rapidly available glucose in the carbohydrate blend comprises a galactose unit and a glucose unit joined by a β (1,4) glycosidic linkage. One example of a carbohydrate that includes a galactose unit and a glucose unit joined by a β (1,4) glycosidic linkage is lactose.

In additional specific embodiments, the source of at least one carbohydrate that provides rapidly available glucose in the carbohydrate blend comprises glucose units joined by α (1,4) glycosidic linkages. Exemplary carbohydrates or carbohydrate sources having glucose units joined by α (1,4) glycosidic linkages include, but are not limited to, maltose, maltodextrin, and starch.

In additional specific embodiments, the source of at least one carbohydrate that provides rapidly available glucose in the carbohydrate blend comprises glucose units joined by α (1,6) glycosidic linkages. One example of a carbohydrate having glucose units joined by α (1,6) glycosidic linkages is isomaltose.

In additional specific embodiments, the source of at least one carbohydrate that provides rapidly available glucose in the carbohydrate blend comprises oligosaccharides having a random mixture of α (1,2), α (1,3), α (1,4), and α (1,6) glycosidic linkages. One example of a source of carbohydrate that includes oligosaccharides having a random mixture of α (1,2), α (1,3), α (1,4), and α (1,6) glycosidic linkages is isomalto-oligosaccharides. Suitable isomalto-oligosaccharides that provide rapidly available glucose include mixtures of oligosaccharides with a degree of polymerization (DP) of 3 or greater including, but not limited to, isomaltose, panose, maltotetraose isomaltotriose, isomaltotetraose, maltopentaose, isomaltopentaose, maltohexaose, isomaltohexaose, maltoheptaose, isomaltoheptaose, maltooctaose, isomaltooctaose, maltononaose, and isomaltononaose.

In an additional embodiment, the source of at least one carbohydrate that provides rapidly available glucose comprises glucose, fructose, tagatose, galactose, mannose, ribose, sucrose, maltose, isomaltose, lactose, isomalto-oligosaccharides, maltodextrin, or starch, or a combination of two or more thereof.

In specific embodiments, the source of at least one carbohydrate in the blend that provides slowly available glucose comprises (i) a glucose unit and a fructose unit joined by an α (1,6) glycosidic linkage; (ii) two glucose units joined by an α (1,1) glycosidic linkage; (iii) a glucose unit and a fructose unit joined by an α (1,5) glycosidic linkage; or (iv) an oligosaccharide having alternating α (1,3) and α (1,6) glycosidic linkages, or a combination of two or more thereof.

In more specific embodiments, the source of at least one carbohydrate that provides slowly available glucose in the carbohydrate blend comprises a glucose unit and a fructose unit joined by an α (1,6) glycosidic linkage. One example of a carbohydrate having a glucose unit and a fructose unit joined by an α (1,6) glycosidic linkage is isomaltulose.

In further specific embodiments, the source of at least one carbohydrate that provides slowly available glucose in the carbohydrate blend comprises two glucose units joined by an α (1,1) glycosidic linkage. One example of a carbohydrate having two glucose units joined by an α (1,1) glycosidic linkage is trehalose.

In additional specific embodiments, the source of at least one carbohydrate that provides slowly available glucose in the carbohydrate blend comprises a glucose unit and a fructose unit joined by an α (1,5) glycosidic linkage. One example of a carbohydrate having a glucose unit and a fructose unit joined by an α (1,5) glycosidic linkage is leucrose. Leucrose is a disaccharide that is present in sucromalt.

In further specific embodiments, the source of at least one carbohydrate that provides slowly available glucose in the carbohydrate blend comprises oligosaccharides having alternating α (1,3) and α (1,6) glycosidic linkages. One example of a source of carbohydrate that includes oligosaccharides having alternating α (1,3) and α (1,6) glycosidic linkages is sucromalt.

In an additional embodiment, the source of at least one carbohydrate that provides slowly available glucose comprises isomaltulose, trehalose, sucromalt, or leucrose, or a combination of two or more thereof.

In specific embodiments, the source of at least one non-digestible carbohydrate or resistant starch included in the carbohydrate blend comprises (i) oligosaccharides having a random mixture of α (1,2), α (1,3), α (1,4), and β glycosidic linkages; (ii) saccharides having linear chains of 2 to 60 fructose units joined by α (2,1) glycosidic linkages or fructose polymers joined by β (2,1) glycosidic linkages; or (iii) oligosaccharides having a random mixture of α (1,2), α (1,3), α (1,4), or α (1,6) glycosidic linkages, or a combination of two or more thereof.

In specific embodiments, the source of at least one non-digestible carbohydrate or resistant starch comprises oligosaccharides having a random mixture of α (1,2), α (1,3), α (1,4), and β glycosidic linkages. One example of a source of carbohydrate that includes oligosaccharides having a random mixture of α (1,2), α (1,3), α (1,4), and β glycosidic linkages is digestion-resistant maltodextrin, or, commonly, resistant maltodextrin. The mixture of oligosaccharides making up resistant maltodextrin are produced by pyrolysis and enzymatic hydrolysis of starch (e.g, corn, wheat, rice, potato) and typically have a molecular weight of about 2,000 Daltons. Examples of commercially available resistant maltodextrin include Nutriose® resistant maltodextrin from Roquette America, Inc. (Geneva, IL) and Fibersol® digestion-resistant maltodextrin from ADM/Matsutani LLC (Itasca, IL).

In additional specific embodiments, the source of at least one non-digestible carbohydrate or resistant starch comprises saccharides having linear chains of 2 to 60 fructose units or fructose polymers joined by β (2,1) glycosidic linkages. Exemplary carbohydrates and carbohydrate sources that include saccharides having linear chains of 2 to 60 fructose units or fructose polymers joined by β (2,1) glycosidic linkages include, but are not limited to, inulin and fructooligosaccharides.

In specific embodiments, the source of at least one non-digestible carbohydrate or resistant starch comprises oligosaccharides having a random mixture of α (1,2), α (1,3), α (1,4), and α (1,6) glycosidic linkages. One example of a source of carbohydrate that includes oligosaccharides having a random mixture of α (1,2), α (1,3), α (1,4), and α (1,6) glycosidic linkages is isomalto-oligosaccharides. The isomalto-oligosaccharides may be any one or more of the isomalto-oligosaccharides previously described herein.

In an additional embodiment, the source of at least one non-digestible carbohydrate or resistant starch comprises resistant maltodextrin, fructooligosaccharides, inulin, or isomalto-oligosaccharides, or a combination of two or more thereof.

In additional embodiments, the carbohydrate blend comprises: (i) a source of at least one carbohydrate that provides rapidly available glucose selected from one or more of glucose, fructose, galactose, mannose, ribose, sucrose, lactose, maltose, isomaltose, maltodextrin, starch, or isomalto-oligosaccharides; (ii) a source of at least one carbohydrate that provides slowly available glucose selected from one or more of isomaltulose, trehalose, leucrose, or sucromalt; and (iii) a source of at least one non-digestible or resistant starch selected from one or more of resistant maltodextrin, fructooligosaccharides, inulin, or isomalto-oligosaccharides. In more specific embodiments, the carbohydrate blend comprises: (i) a source of at least one carbohydrate that provides rapidly available glucose selected from one or more of maltodextrin, isomaltose, or isomalto-oligosaccharides; (ii) a source of at least one carbohydrate that provides slowly available glucose selected from one or more of isomaltulose, sucromalt, trehalose, or leucrose; and (iii) a source of at least one non-digestible or resistant starch selected from one or more of resistant maltodextrin, fructooligosaccharides, inulin, or isomalto-oligosaccharides. In further embodiments, the carbohydrate blend comprises: (i) a source of at least one carbohydrate that provides rapidly available glucose selected from one or more of maltodextrin or isomalto-oligosaccharides; (ii) a source of at least one carbohydrate that provides slowly available glucose selected from one or more of isomaltulose or sucromalt; and (iii) a source of at least one non-digestible or resistant starch selected from one or more of resistant starch, fructooligosaccharides, inulin, or isomalto-oligosaccharides.

Certain sources of carbohydrates used in the carbohydrate blend may include carbohydrates or fractions thereof that provide more than one category of glucose availability (i.e., rapidly available glucose, slowly available glucose, and non-available glucose, i.e., non-digestible carbohydrate or resistant starch). Thus, in accordance with the invention, a source of carbohydrate in the carbohydrate blend can include one or more of a carbohydrate that provides rapidly available glucose, a carbohydrate the provides slowly available glucose, and a non-digestible carbohydrate or resistant starch. For example, a source of carbohydrate that includes a carbohydrate that provides rapidly available glucose and a non-digestible carbohydrate or resistant starch (e.g., through a fiber fraction, which is a non-digestible carbohydrate or resistant starch) is isomalto-oligosaccharides. An example of a source of carbohydrate that includes a carbohydrate that provides rapidly available glucose and a carbohydrate that provides slowly available glucose is sucromalt. Another such example is maltitol, which may be considered a source of carbohydrate that provides slowly available glucose in the carbohydrate blend and also a source of carbohydrate that is non-digestible carbohydrate or resistant starch as its digestion rate is slow and it is not completely digested. Accordingly, a single source of carbohydrate may be used in the methods of the present disclosure to provide one or more than one of a carbohydrate that provides rapidly available glucose, a carbohydrate that provides slowly available glucose, and a non-digestible carbohydrate or resistant starch.

In another specific embodiment, the carbohydrate blend includes fructose, isomaltulose, sucromalt, digestion-resistant maltodextrin, or fructooligosaccharides, or a combination of two or more thereof. In a more specific embodiment, the carbohydrate blend comprises, but is not limited to, fructose, isomaltulose, sucromalt, digestion-resistant maltodextrin, and fructooligosaccharides.

In specific embodiments, the source of at least one carbohydrate of the carbohydrate blend that provides rapidly available glucose provides from 5% to 70% of the total calories supplied by carbohydrates in the nutritional composition, the source of at least one carbohydrate of the carbohydrate blend that provides slowly available glucose provides from 20% to 85% of the total calories supplied by carbohydrates in the nutritional composition, and the source of at least one non-digestible carbohydrate or resistant starch of the carbohydrate blend provides from 5% to 35% of the total calories supplied by carbohydrates in the nutritional composition. In a more specific embodiment, the source of at least one carbohydrate of the carbohydrate blend that provides rapidly available glucose provides from 25% to 60% of the total calories supplied by carbohydrates in the nutritional composition, the source of at least one carbohydrate of the carbohydrate blend that provides slowly available glucose provides from 20% to 50% of the total calories supplied by carbohydrates in the nutritional composition, and the source of at least one non-digestible carbohydrate or resistant starch of the carbohydrate blend provides from 15% to 35% of the total calories supplied by carbohydrates in the nutritional composition.

In a specific embodiment, the BPL1 is administered together with the carbohydrate blend. Whether administered together or separately, the BPL1 and the carbohydrate blend are administered in amounts effective to reduce metabolic alterations which lead to or occur with development of prediabetes and/or diabetes, thereby slowing diabetes or prediabetes development or reducing a risk of developing diabetes in an individual at risk of developing diabetes. In specific embodiments, the BPL1 is administered to the individual in a daily amount of from about 10⁵ to 10¹⁵ cfu, and the carbohydrate blend is administered to the individual in a daily amount of from about 5 to about 100 g, about 5 to about 50 g, or about 5 to about 30 g. In more specific embodiments, the BPL1 and the carbohydrate blend are administered in combination, for example in a nutritional composition or nutritional supplement, in the indicated amounts. In a specific embodiment, the nutritional composition includes protein and fat. In additional embodiments, the BPL1 and the carbohydrate blend are administered at least once daily for a continual period of time, for example at least 7 days, at least 2 weeks, at least 3 weeks, at least 4 weeks, at least 6 weeks, at least 12 weeks, or longer. In further embodiments, the BPL1 and the carbohydrate blend are administered at least once daily as part of routine daily nutrition.

In a specific embodiment of the invention, a nutritional composition comprises protein, fat, the carbohydrate blend, and BPL1. The nutritional composition may be in liquid or powder form. Powder compositions may be consumed as powders or may be reconstituted with liquid, for example, water, for consumption. The concentration and relative amounts of the sources of protein, carbohydrate blend, and fat in the exemplary nutritional compositions can vary considerably depending upon, for example, the specific dietary needs of the intended user. In specific embodiments, the nutritional composition is a powder and comprises from about 10 to about 75 wt %, or about 10 to about 30 wt %, or about 40 to about 75 wt %, protein, from about 5 to about 80 wt %, or about 35 to about 75 wt %, or about 5 to about 15 wt %, carbohydrate blend, and from about 1 to about 30 wt %, or about 5 to 30 wt %, or about 1 to 15 wt %, fat. In another specific embodiment, the nutritional composition is a liquid and comprises from about 1 to about 15 wt %, or about 1 to about 10 wt %, protein, from about 1 to about 25 wt %, or about 5 to about 20 wt %, carbohydrate, and from about 0.1 to about 15 wt %, or about 0.1 to about 10 wt %, fat.

In additional embodiments, the nutritional composition is a powder and comprises from about 10 to about 75 wt % protein, from about 5 to about 80 wt % of the carbohydrate blend, and from about 1 to about 30 wt % fat. In additional embodiments, the powder compositions may comprise from about 10 to about 30 wt % protein, from about 35 to about 75 wt % of the carbohydrate blend, and from about 5 to about 30 wt % fat, or may comprise from about 40 to about 75 wt % protein, from about 5 to about 15 wt % of the carbohydrate blend, and from about 1 to about 15 wt % fat.

In another specific embodiment, the nutritional composition is a liquid and comprises from about 1 to about 15 wt % protein, from about 1 to about 25 wt % carbohydrate blend, and from about 0.1 to about 10 wt % fat, with a major portion of the composition comprising water. In an additional embodiment, the liquid nutritional composition comprises from about 1 to about 10 wt % protein, about 0.1 to about 10 wt % fat, and about 5 to about 20 wt % of the carbohydrate blend.

In a specific embodiment, the nutritional compositions contain BPL1 and the carbohydrate blend in amounts such that a single serving provides sufficient quantities of each to obtain the improvements of the inventive methods. For example, in more specific embodiments, a liquid composition serving size of about 237 ml (8 oz.), prepared as a ready-to-drink liquid or reconstituted from powder, comprises from about 10⁵ to 10¹⁵ cfu BPL1, or more specifically, from about 10⁸ to 10¹² cfu BPL1, and from about 5 to about 100 g, about 5 to about 50 g, or about 5 to about 30 g, of the carbohydrate blend.

In additional specific embodiments, which render the compositions suitable for use in accordance with specific embodiments of the inventive methods discussed herein, the carbohydrate blend of the nutritional compositions comprises fructose, isomaltulose, sucromalt, digestion-resistant maltodextrin, fructooligosaccharides, or a combination of two or more thereof. In a more specific embodiment of the nutritional compositions, the carbohydrate blend comprises, but is not limited to, fructose, isomaltulose, sucromalt, digestion-resistant maltodextrin, and fructooligosaccharides.

The protein which is contained in the nutritional composition may be any one or more proteins known for use in nutritional compositions. A wide variety of sources and types of protein can be used in the nutritional compositions. For example, the source of protein may include, but is not limited to, intact, hydrolyzed, and partially hydrolyzed protein, which may be derived from any suitable source such as milk (e.g., casein, whey), animal (e.g., meat, fish), cereal (e.g., rice, brown rice, corn, barley, etc.), vegetable (e.g., soy, pea, yellow pea, fava bean, chickpea, canola, potato, mung, ancient grains such as quinoa, amaranth, and chia, hemp, flax seed, etc.), and combinations of two or more thereof. The protein may also include one or a mixture of naturally occurring or synthetic amino acids (often described as free amino acids) and/or their metabolites, known for use in nutritional products, alone or in combination with the intact, hydrolyzed, and/or partially hydrolyzed proteins described herein.

More specific examples of sources of protein which are suitable for use in the nutritional compositions described herein include, but are not limited to, whey protein, whey protein concentrates, whey protein isolates, whey protein hydrolysates, acid caseins, casein protein isolates, sodium caseinates, calcium caseinates, potassium caseinates, casein hydrolysates, milk protein concentrates, milk protein isolates, milk protein hydrolysates, nonfat dry milk, condensed skim milk, whole cow's milk, partially or completely defatted milk, whole egg powder, egg yolk powder, egg white powder, coconut milk, soy protein concentrates, soy protein isolates, soy protein hydrolysates, pea protein concentrates, pea protein isolates, pea protein hydrolysates, rice protein concentrate, rice protein isolate, rice protein hydrolysate, barley rice protein, fava bean protein concentrate, fava bean protein isolate, fava bean protein hydrolysate, collagen proteins, collagen protein isolates, meat proteins such as beef protein isolate and/or chicken protein isolate, potato proteins, chickpea proteins, canola proteins, mung proteins, quinoa proteins, amaranth proteins, chia proteins, hemp proteins, flax seed proteins, earthworm proteins, insect proteins, and combinations of two or more thereof. The nutritional compositions can include any individual source of protein or a combination of any two or more sources of protein. In specific embodiments, the nutritional compositions comprise at least one milk protein, or at least one plant protein, or at least one milk protein and at least one plant protein. In additional embodiments, the protein comprises caseinate, for example, sodium caseinate, calcium caseinate, potassium caseinate, or casein hydrolysate, milk protein concentrate, soy protein, more specifically soy protein isolate, or a combination of two or more thereof.

The inventive nutritional compositions also include fat. The term “fat” as used herein, unless otherwise specified, refers to lipids, fats, oils, and combinations thereof. Sources of fat suitable for use in the nutritional composition include, but are not limited to, algal oil, canola oil, flaxseed oil, borage oil, safflower oil, high oleic safflower oil, high gamma-linolenic acid (GLA) safflower oil, corn oil, soy oil, sunflower oil, high oleic sunflower oil, cottonseed oil, coconut oil, fractionated coconut oil, medium chain triglycerides (MCT) oil, palm oil, palm kernel oil, palm olein, lecithin, and long chain polyunsaturated fatty acids such as docosahexanoic acid (DHA), arachidonic acid (ARA), docosapentaenoic acid (DPA), eicosapentaenoic acid (EPA), and combinations thereof. The nutritional compositions can include any individual source of fat or a combination of two or more sources of fat. In a specific embodiment, the fat comprises high oleic sunflower oil, soy oil, or a combination thereof.

In specific embodiments, the nutritional composition in liquid form, either prepared as a ready to drink liquid or reconstituted to liquid form from a powder composition, and has a neutral pH, i.e., a pH of from about 6 to 8 or, more specifically, from about 6 to 7.5. In more specific embodiments, the nutritional composition has a pH of from about 6.5 to 7.2 or, more specifically, from about 6.8 to 7.1.

The nutritional composition may further comprise one or more additional components that may modify the physical, chemical, aesthetic, or processing characteristics of the nutritional composition or serve as additional nutritional components. Non-limiting examples of additional components include preservatives, emulsifying agents (e.g., lecithin), buffers, sweeteners including artificial sweeteners (e.g., saccharine, aspartame, acesulfame K, sucralose), natural sweeteners, colorants, flavorants, thickening agents, stabilizers, and so forth. In a specific embodiment, the nutritional compositions include myo-inositol, which may increase insulin sensitivity. In a specific embodiment, the nutritional compositions may comprise from about 0.1 to about 5 wt % myo-inositol.

Additionally, the nutritional composition may further include vitamins or related nutrients, non-limiting examples of which include vitamin A, vitamin B12, vitamin C, vitamin D, vitamin K, thiamine, riboflavin, pyridoxine, niacin, folic acid, pantothenic acid, biotin, choline, inositol, salts and derivatives thereof, and combinations thereof. Water soluble vitamins may be added in the form of a water-soluble vitamin (WSV) premix and/or oil-soluble vitamins may be added in one or more oil carriers as desired.

In additional embodiments, the nutritional composition may further include one or more minerals, non-limiting examples of which include calcium, phosphorus, magnesium, zinc, manganese, sodium, potassium, molybdenum, chromium, chloride, and combinations thereof.

The nutritional composition may be formed using any techniques known in the art. In one embodiment, the nutritional composition may be formed by (a) preparing an aqueous solution comprising protein and carbohydrate; (b) preparing an oil blend comprising fat and oil-soluble components; and (c) mixing together the aqueous solution and the oil blend to form an emulsified liquid nutritional composition. The BPL1 may be added at any time as desired in the process, for example, to the aqueous solution or to the emulsified blend. The composition may be spray-dried or otherwise dried if a powder product is desirable. Alternatively, a powder product can be formed by dry blending powdered ingredients.

The following Example demonstrates aspects of the inventive methods and compositions.

### EXAMPLES

The following examples demonstrate the beneficial effects of BPL1 in combination with the described carbohydrate blend in the management of metabolic alterations associated with diabetes evolution. Results in the examples are expressed as mean±standard error of the mean (SEM). Statistical analysis was performed using one-way analysis of variance. Multiple comparisons of means were done by the Fisher's LSD test. p<0.05 was considered statistically significant.

The examples used heat-killed BPL1 (BPL1 HT) from Archer Daniels Midland Company (ADM) (Chicago, IL USA) and was obtained by ADM as described Caimari et al., Journal Functional Foods (2017), 38: 251-63. Briefly, in the process disclosed by Caimari et al., the BPL1 strain was isolated from feces of healthy babies undergoing breast-milk feeding. The BPL1 strain was grown anaerobically and then inactivated by heat treatment (autoclaved at 121° C. for 20 min), harvested by centrifugation, mixed with maltodextrin and lyophilized. Once obtained, the heat-killed BPL1 powder was standardized taking into account the total cfu content obtained in culture and the grams of powder recovered by combining with maltodextrin.

Wistar rats weighing 200-250 g, purchased from Envigo (France) were employed in the examples. The animals were housed in a temperature-controlled room at 22+2° C., with 50±10% humidity and a 12-h dark/12-h light cycle and, prior to the experiments described herein, were provided with standard rodent diet AlN93M and demineralized water adlibitum.

A rat model of type 2 diabetes that replicates the natural history and metabolic characteristic of human type 2 diabetes was employed. In this rat model, the diabetes pattern is achieved by combining (1) feeding of a high fat (HF) diet which produces obesity and insulin resistance, and (2) streptozotocin (STZ) treatment that causes initial beta cell dysfunction and subsequent hyperglycemia. More specifically, feeding of the high fat diet to rats for a period of 10 weeks produces obese rats as employed in Example 1 with a condition similar to prediabetic state in humans that is characterized by insulin-resistant obesity, mild hyperglycemia, hypertriglyceridemia, hypercholesterolemia and compensatory hyperinsulinemia together with reduced glucose disappearance rate. The evolution of a diabetic disease pattern was achieved in the insulin-resistant rats upon injection with a low dose of STZ which produced frank hyperglycemia as described in more detail in Example 2.

### Example 1

This example demonstrates the improvements provided by the methods and compositions of the invention on obese subjects which had been fed a high fat (HF) diet for 10 weeks prior to the start of this experiment. More specifically, the obese high-fat fed rats as described above were employed in this example to assess the effect of administration of BPL1 (heat treated, BPL1 HT) and the carbohydrate blend (CBLEND) to obese rats. The obese rats were divided into four groups for diet administration with the macronutrient profiles of the respective diets shown in Table 1:
OB group, continued on HF diet, no additional supplement.
OB+BPL1 HT group, continued on HF diet, supplemented with 10¹⁰ cfu/day of the heat-treated BPL1.
OB+CBLEND group, continued on HF diet, supplemented with the carbohydrate blend described in Table 1 (CBLEND) in place of conventional carbohydrates sucrose and corn starch.
OB+BPL1 HT+CBLEND group, continued on HF diet, supplemented with 10¹⁰ cfu/day of the heat-killed BPL1 and the carbohydrate blend in place of conventional carbohydrates sucrose and corn starch.

The obese animals received their respective diets for a period of 4 weeks. Animals in a lean control group (Lean group) were fed AlN93M throughout the study, including the initial 10 week feeding.

TABLE 1
Macronutrient Profiles of Diets

| | | | | | HF + |
|---|---|---|---|---|---|
| | Lean | | HF + | HF + | BPL1HT + |
| Macronutrient | (AIN93M) | HF | BPL1HT | CBLEND | CBLEND |
| CHO (g/100 g diet) | 75.7 | 44.7 | 44.8 | 42.2 | 42.2 |
| Sucrose (g/100 g CHO) | 13.5 | 48.2 | 48.2 | | |
| Fructose (g/100 g CHO) | | | | 7.6 | 7.6 |
| Isomaltulose (g/100 g CHO) | | | | 5.4 | 5.4 |
| Sucromalt (g/100 g CHO) | | | | 11.8 | 11.8 |
| Corn Starch (g/100 g CHO) | 59.8 | 28.9 | 28.9 | | |
| Maltodextrins (g/100 g CHO) | 19.9 | 14.5 | 14.1 | 32.6 | 32.6 |
| Resistant maltodextrin (g/100 g CHO) | | | | 21.0 | 21.0 |
| Soluble fiber (g/100 g CHO) | | | | 5.2 | 5.2 |
| Insoluble fiber (g/100 g CHO) | 6.8 | 8.3 | 8.3 | 2.0 | 2.0 |
| Maltitol (g/100 g CHO) | | | | 12.7 | 12.7 |
| Protein (g/100 g diet) | 12.2 | 22.6 | 22.6 | 22.6 | 22.6 |
| Fat (g/100g diet) | 4.00 | 19.3 | 19.3 | 19.3 | 19.3 |
| Myo-inositol (g/100 g diet) | | | | 1.81 | 1.81 |
| Heat-killed BPL1 (cfu/100 g diet) | | | 5.1 × 10¹⁰ | | 5.1 × 10¹⁰ |

The protein source in all diets was calcium caseinate. The fat source in the AlN93M lean diet comprised soy fat, and the fat source in the high fat (HF) diets comprised lard fat.

Body composition was measured using magnetic resonance imaging (EchoMRI system, EchoMRI, Houston, TX, USA) at the end of the 4 week feeding period (day 28). In addition, blood samples were collected postprandial for analyzing glucagon-like peptide-1 (GLP-1) in plasma using an ELISA kit (Mercodia, Uppsala, Sweden). Cecum was also collected, weighed and frozen at the day 28 of the study for evaluating short-chain fatty acids (SCFAs) in the cecum. SCFAs were derivatized and analyzed by liquid chromatography-tandem mass spectrometry (LC-MS/MS). Total SCFAs were quantified in cecum, and the results were expressed in μg of wet weight cecum content, as described by Zeng et al., J Chrom B Analyt Technol Biomed Life Sci (2018), 1083: 137-145.

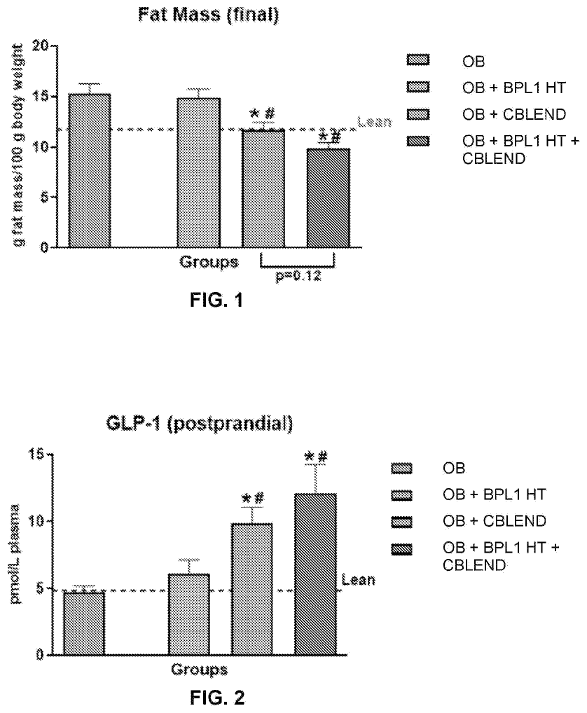

FIG. 1 shows the amount of fat mass of the respective groups of animals in g fat mass/100 g body weight. Animals from the OB group showed a higher amount of fat mass in comparison with animals from the Lean group. When the OB group was compared to the OB+BPL1 HT group, no significant difference was observed. In contrast, both the OB+CBLEND group and the OB+BPL1 HT+CBLEND group had significantly lower fat contents than the OB and OB+BPL1 HT groups. Significantly, the OB+BPL1 HT+CBLEND group exhibited the lowest fat mass content as compared to the rest of the obese treated groups, and, more specifically, the OB+BPL1 HT+CBLEND group exhibited 36%, 34% and 15% less fat mass than the OB, OB+BPL1 HT and OB+CBLEND groups, respectively. In FIG. 1, a p value<0.05 was considered significant; (*) shows statistical significance vs the OB group, and (#) shows statistical significance vs the OB+BPL1 HT group. The difference between the OB+CBLEND group and the OB+BPL1 HT+CBLEND group is indicated with the p value under the columns.

GLP-1 is an incretin which is synthesized and released by intestinal L-cells, with nutrient intake as the primary stimulus. GLP-1 has been shown to suppresses appetite in both normal and obese individuals, playing a major role in energy metabolism regulation. As shown in FIG. 2, in this experiment, GLP-1 secretion was not affected by the HF diet, showing similar levels in the OB group and the Lean group. The addition of BPL1 HT to the HF diet also had no significant effect on GLP-1 secretion by the OB+BPL1 HT group. However, GLP-1 levels in the OB+CBLEND group and the OB+BPL1 HT+CBLEND were significantly higher than in the other two groups. Significantly, in the OB+BPL1 HT+CBLEND group, the combination of slow digestible carbohydrate and BPL1 HT showed the highest concentration of GLP-1 in plasma, 23% more than the observed in the OB+CBLEND. In FIG. 2, a p value<0.05 was considered significant; (*) shows statistical significance vs the OB group, and (#) shows statistical significance vs the OB+BPL1 HT group.

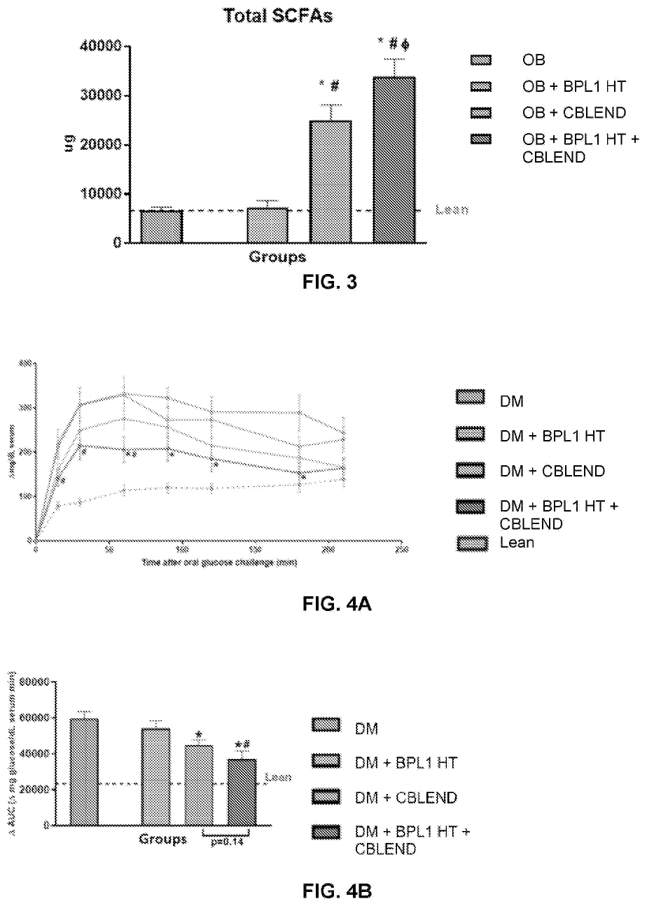

An increase in the production of SCFAs, due to fermentation of non-digestible carbohydrates, has been demonstrated to enhance activation of L cells in the intestine and increase GLP-1 secretion. As shown in FIG. 3, when total SCFAs were analyzed in the cecum samples, an increase in the OB+CBLEND group achieving statistical significance in comparison to the OB group and the OB+BPL1 HT group was observed. However, the highest amount of total SCFAs was observed in the OB+BPL1 HT+CBLEND group, which was significantly higher than the other three groups. In FIG. 3, a p value<0.05 was considered significant; (*) shows statistical significance vs the OB group, (#) shows statistical significance vs the OB+BPL1 HT group, and ($) shows statistical significance vs the OB+CBLEND group.

In summary, the combination of BPL1 HT and the carbohydrate blend provided reduction in fat body mass, increase in postprandial GLP-1, and increase in total cecum SCFAs as compared with BPL1 HT individually and as compared with the carbohydrate blend individually, as follows:

TABLE 2
Improvements of BPL1 HT + CBLEND
over Individual Components in Obese Subjects

| Obese Condition | BPL1 HT alone | CBLEND alone |
|---|---|---|
| Fat body mass | ↓ 34% | ↓ 15% |
| Postprandial GLP-1 | ↑ 97% | ↑ 23% |
| Cecum SCFAs | ↑ 365% | ↑ 36% |

These results show that the combination of BPL1 and the carbohydrate blend can reduce metabolic conditions associated with the development of diabetes and prediabetes in obese subjects.

### Example 2

This example shows the effect of BLP1 and the carbohydrate blend on the regulation of diabetes progression. Rats were fed the respective diets as shown in Table 1. After 4 weeks of feeding, the obese rats which had been fed the respective experimental diets as described in Example 1 (HF-containing diet with or without BPL1 HF and/or CBLEND) were fasted for 12 h to induce a decline in secretory capacity of pancreatic beta cells to compensate for the existing insulin resistance and then administered a single dose of STZ in a citrate buffer (50 mmol/L; pH 4.5) intraperitoneally (30 mg/kg body weight) to induce a diabetic condition.

The respective diets of the diabetic animal groups were those shown in Table 1, and are designated as follows:
DM group, continued on HF diet, no additional supplement.
DM+BPL1 HT group, continued on HF diet, supplemented with 10¹⁰ cfu/day of the heat-killed BPL1.
DM+CBLEND group, continued on HF diet, supplemented with the carbohydrate blend in place of conventional carbohydrates sucrose and corn starch.
DM+BPL1 HT+CBLEND group, continued on HF diet, supplemented with 10¹⁰ cfu/day of the heat-killed BPL1 and the carbohydrate blend in place of conventional carbohydrates sucrose and corn starch.

Animals in a lean control group (Lean group) were fed AlN93M throughout the study.

The animals continued on the respective diets for four weeks after STZ injection, and were then subjected to an oral glucose tolerance test (OGTT). Diabetic animals that had been fasting for 12 hours received a glucose load of 2.5 g/kg body weight. The glucose load was administered through an orogastric gavage. Blood samples were collected before (t=0) and after the glucose administration (15, 30, 60, 90, 120, and 180 min). Once the test was completed, blood HbA1c was measured with a clinical chemistry analyzer (Pentra 400, Horiba ABX, Montpellier, France).

FIGS. 4A and 4B respectively show the glucose response as a function of time and the area under the curve (AUC). As shown in FIG. 4A, the Lean group showed a lower postprandial glucose response in comparison with the rest of the groups, while DM and DM+BPL1 HT group showed the highest responses. In contrast, the DM+BPL1 HT+CBLEND group showed the lowest glycemic response of the diabetic groups throughout the measured time period. FIG. 4B shows that the AUC for the DM+CBLEND group was significantly lower as compared to the DM group, although no statistical difference was seen between the DM+CBLEND group and the DM+BPL1 HT. The lowest value of AUC was obtained in the DM+BPL1 HT+CBLEND group, which was significantly lower than both the DM group and the DM+BPL1 HT group, as well as being 17% lower than DM+CBLEND group. In FIG. 4B, a p value<0.05 was considered significant; (*) shows statistical significance vs the DM group, (#) shows statistical significance vs the DM+BPL1 HT group. The difference between the DM+CBLEND group and the DM+BPL1 HT+CBLEND group is indicated with the p value under the columns.

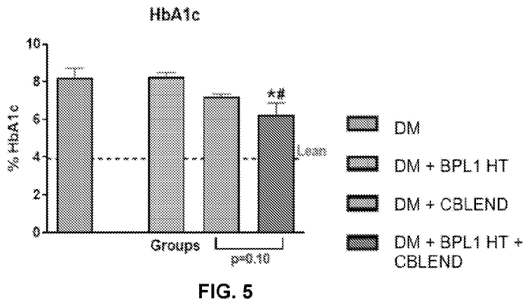

In relation to blood glucose levels, the glycosylated form of hemoglobin (HbA1c) has been recognized as a diagnostic tool, increasing with the progression of diabetes. In this experiment, an increase of HbA1c was observed in all groups relative to the Lean group. As shown in FIG. 5, when the diabetic groups were compared, the lowest value of HbA1c was obtained in the DM+BPL1 HT+CBLEND group, and the value was significantly different from both the DM group and the DM+BPL1 HT group, and also tended to be lower (p=0.10) in comparison with DM+CBLEND group. The DM+BPL1 HT+CBLEND group had 2%, 2% and 1% less HbA1c than the DM, DM+BPL1 HT and DM+CBLEND groups, respectively. Those skilled in the art appreciate that these decreases have an important relevance. For example, each 1% reduction in mean HbA1c has been associated with a reduction in diabetes-related deaths by 21%, a reduction in myocardial infarction by 14%, and a reduction in microvascular complications by 14%. (See Stratton et al. (2000) Br Med J, 321: 405-12). In FIG. 5, a p value<0.05 was considered significant; (*) shows statistical significance vs the DM group, (#) shows statistical significance vs the DM+BPL1 HT group. The difference between the DM+CBLEND group and the DM+BPL1 HT+CBLEND group is indicated with the p value under the columns.

In summary, the combination of BPL1 HT and the carbohydrate blend provided reductions in both the postprandial glucose response and in blood HbA1c as compared with BPL1 HT individually and as compared with the carbohydrate blend individually, as follows:

TABLE 3
Improvements of BPL1 HT + CBLEND over
Individual Components in Diabetic Subjects

| Diabetic Condition | BPL1 HT alone | CBLEND alone |
|---|---|---|
| Postprandial glucose response | ↓ 31% | ↓ 17% |
| Blood HbA1c content | ↓ 2% | ↓ 1% |

These results show that the combination of BPL1 and the carbohydrate blend can reduce metabolic conditions associated with the development of diabetes in diabetic subjects.

While the present application has been illustrated by the description of embodiments and examples thereof, and while the embodiments and examples have been described in considerable detail, such descriptions are not intended to restrict or in any way limit the scope of the appended claims to such detail. Additional advantages and modifications will readily appear to those skilled in the art. Therefore, the invention, in its broader aspects, is not limited to the specific details, the representative methods or compositions, or illustrative examples shown and described. Accordingly, departures may be made from such details without departing from the spirit or scope of the general inventive concept.

What is claimed is:

1. A method for slowing diabetes development in a diabetic or prediabetic individual or reducing a risk of diabetes development in an individual at risk of developing diabetes, the method comprising administering Bifidobacterium animalis subsp. lactis CECT 8145 (BPL1) and a carbohydrate blend to the individual, wherein the carbohydrate blend comprises a source of at least one carbohydrate that provides rapidly available glucose, a source of at least one carbohydrate that provides slowly available glucose, and a source of at least one non-digestible carbohydrate or resistant starch.

2. The method of claim 1, wherein the source of at least one carbohydrate that provides rapidly available glucose comprises:
(i) a monosaccharide;
(ii) a glucose unit and a fructose unit joined by an α-1, β-2 glycosidic linkage;
(iii) a glucose unit and a galactose unit joined by a β (1,4) glycosidic linkage;
(iv) glucose units joined by α (1,4) glycosidic linkages;
(v) glucose units joined by α (1,6) glycosidic linkages; or
(vi) oligosaccharides having a random mixture of α (1,2), α (1,3), α (1,4), and α (1,6) glycosidic linkages;
or a combination of two or more thereof.

3. The method of claim 1 or 2, wherein the source of at least one carbohydrate that provides rapidly available glucose comprises glucose, fructose, tagatose, galactose, mannose, ribose, sucrose, maltose, isomaltose, lactose, isomalto-oligosaccharides, maltodextrin, or starch, or a combination of two or more thereof.

4. The method of any one of claims 1-3, wherein the source of at least one carbohydrate that provides slowly available glucose comprises (i) a glucose unit and a fructose unit joined by an α (1,6) glycosidic linkage; (ii) two glucose units joined by an α (1,1) glycosidic linkage; (iii) a glucose unit and a fructose unit joined by an α (1,5) glycosidic linkage; or (iv) an oligosaccharide having alternating α (1,3) and α (1,6) glycosidic linkages, or a combination of two or more thereof.

5. The method of any one of claims 1-4, wherein the source of at least one carbohydrate that provides slowly available glucose comprises isomaltulose, trehalose, sucromalt, or leucrose, or a combination of two or more thereof.

6. The method of any one of claims 1-5, wherein the source of at least one non-digestible carbohydrate or resistant starch comprises:
(i) oligosaccharides having a random mixture of α (1,2), α (1,3), α (1,4), and β glycosidic linkages;
(ii) saccharides having linear chains of 2 to 60 fructose units or fructose polymers joined by β (2,1) glycosidic linkages; or
(iii) oligosaccharides having a random mixture of α (1,2), α (1,3), α (1,4), and α (1,6) glycosidic linkages,
or a combination of two or more thereof.

7. The method of any one of claims 1-6, wherein the source of at least one non-digestible carbohydrate or resistant starch comprises resistant maltodextrin, fructooligosaccharides, inulin, or isomalto-oligosaccharides, or a combination of two or more thereof.

8. The method of claim 1, wherein the carbohydrate blend comprises fructose, isomaltulose, sucromalt, digestion-resistant maltodextrin, fructooligosaccharides, or a combination of two or more thereof.

9. The method of claim 1, wherein the carbohydrate blend comprises fructose, isomaltulose, sucromalt, digestion-resistant maltodextrin, and fructooligosaccharides.

10. The method of any one of claims 1-9, wherein the source of at least one carbohydrate that provides rapidly available glucose provides from 5% to 70%, or 25% to 60%, of the total calories supplied by carbohydrates in the nutritional composition, the source of at least one carbohydrate of the carbohydrate blend that provides slowly available glucose provides from 20% to 85%, or 20% to 50%, of the total calories supplied by carbohydrates in the nutritional composition, and the source of at least one non-digestible carbohydrate or resistant starch of the carbohydrate blend provides from 5% to 35% or 15% to 35%, of the total calories supplied by the carbohydrate blend.

11. The method of any one of claims 1-10, wherein the BPL1 is inanimate.

12. The method of any one of claims 1-11, wherein the individual is obese.

13. The method of any one of claims 1-12, wherein the individual exhibits insulin resistance.

14. The method of any one of claims 1-13, wherein the individual exhibits prediabetes or diabetes.

15. The method of any one of claims 1-14, wherein the BPL1 is administered to the individual in a daily amount of from about 10⁵ to 10¹⁵ cfu BPL1, or from about 10⁸ to 10¹² cfu BPL1.

16. The method of claim 15, wherein the daily amount of BPL1 is administered with from about 5 to about 100 g, about 5 to about 50 g, or about 5 to about 30 g, of the carbohydrate blend.

17. The method of any one of claims 1-16, wherein the BPL1 and the carbohydrate blend are administered to the individual at least once daily for a period of at least 7 days.

18. The method of any one of claims 1-17, wherein the BPL1 and the carbohydrate blend are administered to the individual in a nutritional composition further comprising protein and fat.

19. A nutritional composition, comprising protein, fat, a carbohydrate blend, and Bifidobacterium animalis subsp. lactis CECT 8145 (BPL1), wherein the carbohydrate blend comprises a source of at least one carbohydrate that provides rapidly available glucose, a source of at least one carbohydrate that provides slowly available glucose, and a source of at least one non-digestible carbohydrate or resistant starch.

20. The nutritional composition of claim 19, wherein the source of at least one carbohydrate that provides rapidly available glucose comprises:
(i) a monosaccharide;
(ii) a glucose unit and a fructose unit joined by an α-1, β-2 glycosidic linkage;
(iii) a glucose unit and a galactose unit joined by a β (1,4) glycosidic linkage;
(iv) glucose units joined by α (1,4) glycosidic linkages;
(v) glucose units joined by α (1,6) glycosidic linkages; or
(vi) oligosaccharides having a random mixture of α (1,2), α (1,3), α (1,4), and α (1,6) glycosidic linkages;
or a combination of two or more thereof.

21. The nutritional composition of claim 20, wherein the source of at least one carbohydrate that provides rapidly available glucose comprises glucose, fructose, tagatose, galactose, mannose, ribose, sucrose, maltose, isomaltose, lactose, isomalto-oligosaccharides, maltodextrin, or starch, or a combination of two or more thereof.

22. The method of any one of claims 19-21, wherein the source of at least one carbohydrate that provides slowly available glucose comprises (i) a glucose unit and a fructose unit joined by an α (1,6) glycosidic linkage; (ii) two glucose units joined by an α (1,1) glycosidic linkage; (iii) a glucose unit and a fructose unit joined by an α (1,5) glycosidic linkage; or (iv) an oligosaccharide having alternating α (1,3) and α (1,6) glycosidic linkages, or a combination of two or more thereof.

23. The nutritional composition of any one of claims 19-22, wherein the source of at least one carbohydrate that provides slowly available glucose comprises isomaltulose, trehalose, sucromalt, or leucrose, or a combination of two or more thereof.

24. The nutritional composition of any one of claims 19-23, wherein the source of at least one non-digestible carbohydrate or resistant starch comprises:
(i) oligosaccharides having a random mixture of α (1,2), α (1,3), α (1,4), and β glycosidic linkages;
(ii) saccharides having linear chains of 2 to 60 fructose units or fructose polymers joined by β (2,1) glycosidic linkages; or
(iii) oligosaccharides having a random mixture of α (1,2), α (1,3), α (1,4), and α (1,6) glycosidic linkages,
or a combination of two or more thereof.

25. The nutritional composition of claim 24, wherein the source of at least one non-digestible carbohydrate or resistant starch comprises resistant maltodextrin, fructooligosaccharides, inulin, or isomalto-oligosaccharides, or a combination of two or more thereof.

26. The nutritional composition of claim 19, wherein the carbohydrate blend comprises fructose, isomaltulose, sucromalt, digestion-resistant maltodextrin, fructooligosaccharides, or a combination of two or more thereof.

27. The nutritional composition of claim 19, wherein the carbohydrate blend comprises fructose, isomaltulose, sucromalt, digestion-resistant maltodextrin, and fructooligosaccharides.

28. The nutritional composition of any one of claims 19-27, wherein the source of at least one carbohydrate that provides rapidly available glucose provides from 5% to 70%, or 25% to 60%, of the total calories supplied by carbohydrates in the nutritional composition, the source of at least one carbohydrate of the carbohydrate blend that provides slowly available glucose provides from 20% to 85%, or 20% to 50%, of the total calories supplied by carbohydrates in the nutritional composition, and the source of at least one non-digestible carbohydrate or resistant starch of the carbohydrate blend provides from 5% to 35% or 15% to 35%, of the total calories supplied by the carbohydrate blend.

29. The nutritional composition of any one of claims 19-28, wherein the nutritional composition is a powder and comprises from about 10 to about 75 wt % protein, from about 1 to about 30 wt % fat, and from about 5 to about 80 wt % of the carbohydrate blend; or from about 10 to about 30 wt % protein, from about 5 to about 30 wt % fat, and from about 35 to about 75 wt % of the carbohydrate blend; or from about 40 to about 75 wt % protein, from about 1 to about 15 wt % fat, and from about 5 to about 15 wt % of the carbohydrate blend.

30. The nutritional composition of any one of claims 19-28, wherein the nutritional composition is a liquid and comprises from about 1 to about 15 wt % protein, from about 0.1 to about 15 wt % fat, and from about 1 to about 25 wt % of the carbohydrate blend; or from about 1 to about 10 wt % protein, from about 0.1 to about 10 wt % fat, and from about 5 to about 20 wt % of the carbohydrate blend.

31. The nutritional composition of any one of claims 19-30, wherein the BPL1 is inanimate.

32. The nutritional composition of any one of claims 19-31, wherein the protein comprises whey protein, whey protein concentrate, whey protein isolate, whey protein hydrolysate, acid casein, casein protein isolate, sodium caseinate, calcium caseinate, potassium caseinate, casein hydrolysate, milk protein concentrate, milk protein isolate, milk protein hydrolysate, nonfat dry milk, condensed skim milk, whole cow's milk, partially or completely defatted milk, whole egg powder, egg yolk powder, egg white powder, coconut milk, soy protein concentrate, soy protein isolate, soy protein hydrolysate, pea protein concentrates pea protein isolate, pea protein hydrolysate, rice protein concentrate, rice protein isolate, rice protein hydrolysate, barley rice protein, fava bean protein concentrate, fava bean protein isolate, fava bean protein hydrolysate, collagen protein, collagen protein isolate, meat protein, potato protein, chickpea protein, canola protein, mung protein, quinoa protein, amaranth protein, chia protein, hemp protein, flax seed protein, earthworm protein, insect protein, or a combination of two or more thereof, or the protein comprises a caseinate, milk protein concentrate, soy protein, or a combination of two or more thereof.

33. The nutritional composition of any one of claims 19-32, wherein the fat comprises algal oil, canola oil, flaxseed oil, borage oil, safflower oil, high oleic safflower oil, high gamma-linolenic acid (GLA) safflower oil, corn oil, soy oil, sunflower oil, high oleic sunflower oil, cottonseed oil, coconut oil, fractionated coconut oil, medium chain triglycerides (MCT) oil, palm oil, palm kernel oil, palm olein, lecithin, long chain polyunsaturated fatty acid, or a combination of two or more thereof, or comprises high oleic sunflower oil, soy oil, or a combination thereof.

34. The nutritional composition of any one of claims 19-33, further comprising myo-inositol.

35. The nutritional composition of any one of claims 19-34, wherein a liquid composition serving size of about 237 ml comprises from about 10⁵ to 10¹⁵ cfu BPL1, or from about 10⁸ to 10¹² cfu BPL1, and from about 5 to about 100 g, about 5 to about 50 g, or about 5 to about 30 g, of the carbohydrate blend.
